(19) United States
(12) Patent Application Publication
(10) Pub. No.: US 2019/0224347 A1
(43) Pub. Date: Jul. 25, 2019

(54) Detection of Renal Amyloidosis by Imaging with Stilbene or Phenylbenzothiazole Derivatives

(71) Applicant: Mayo Foundation for Medical Education and Research, Rochester, MN (US)

(72) Inventors: Geoffrey B. Johnson, Rochester, MN (US); Bradley J. Kemp, Rochester, MN (US); Nelson Leung, Rochester, MN (US); Samih H. Nasr, Rochester, MN (US); Marina Ramirez-Alvarado, Rochester, MN (US)

(21) Appl. No.: 16/254,163

(22) Filed: Jan. 22, 2019

Related U.S. Application Data
(60) Provisional application No. 62/619,193, filed on Jan. 19, 2018.

(51) Int. Cl. A61K 51/04

(52) U.S. Cl. CPC A61K 51/0455

(57) ABSTRACT

A method for detecting or ruling out renal amyloidosis in a subject is disclosed. The use of a stilbene derivative, such as florbetapir ¹⁸F, or a phenylbenzothiazole derivative in positron emission tomography imaging, or integrated positron emission tomography/computed tomography imaging, can serve as a screening test for renal amyloidosis in a subject.

### CROSS-REFERENCES TO RELATED APPLICATIONS

This application claims priority to U.S. Patent Application No. 62/619,193 filed Jan. 19, 2018, which is incorporated by reference as if set forth in its entirety.

### STATEMENT REGARDING FEDERALLY SPONSORED RESEARCH

Not Applicable.

### BACKGROUND OF THE INVENTION

### 1. Field of the Invention

The invention relates to a method for detecting or ruling out renal amyloidosis in a subject using a stilbene derivative or a phenylbenzothiazole derivative, and a medical imaging technique, such as positron emission tomography imaging, or positron emission tomography with computed tomography imaging.

### 2. Description of the Related Art

Amyloidosis is the abnormal deposition and accumulation of insoluble protein fibrils in organs or tissues. The two most common types of renal amyloidosis are immunoglobulin light chain-derived amyloidosis (AL) and reactive (secondary) amyloidosis (AA). LECT2 amyloidosis (ALECT2) is a form of amyloidosis caused by the leucocyte cell-derived chemotaxin-2 (LECT2) protein. ALECT2 is now recognized as the third most common type of renal amyloidosis in the United States, predominately in subjects of Mexican or Native American descent. Cases have also been described in subjects of Egyptian, Indian and Arab descent. The lack of cardiac or nerve involvement makes LECT2 amyloidosis difficult to recognize and no laboratory test currently exists for screening.

What is needed therefore is a method for detecting or ruling out renal amyloidosis in a subject. In particular, there is a need for a method for detecting or ruling out accumulation of insoluble protein structures in the kidney caused by leucocyte cell-derived chemotaxin-2 (LECT2) protein.

### SUMMARY OF THE INVENTION

This disclosure provides a method for detecting or ruling out renal amyloidosis in a subject. This disclosure also provides a method for detecting or ruling out accumulation of insoluble protein structures in the kidney caused by leucocyte cell-derived chemotaxin-2 (LECT2) protein.

In one version of the method, a detectable amount of a compound of formula (I):

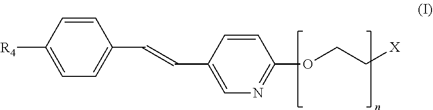

wherein R₄ is selected from the group consisting of H, OH, halogen, substituted or unsubstituted alkyl, substituted or unsubstituted alkoxy, substituted or unsubstituted amino, and substituted or unsubstituted carboxylate,

wherein n is an integer from 0 to 10,

wherein X is selected from the group consisting of ³H, ¹¹C, ¹³C, ¹⁴C, ¹³N, ¹⁵O, ⁷⁵Br, ⁷⁶Br, ¹⁸F, ¹⁹F, ⁶⁸Ga, ⁸²Rb, ¹¹¹In, ¹²³I, ¹²⁵I, ¹³¹I, ²⁰¹TI, and ^{99m}Tc, is administered to a subject. The compound is targeted to any amyloidosis in a kidney of the subject. An image is then acquired to detect the presence or absence of any amyloidosis in a kidney of the subject. The step of acquiring the image can be performed using an imaging method selected from positron emission tomography (PET) imaging, positron emission tomography with concurrent computed tomography (CT) imaging, and positron emission tomography with concurrent magnetic resonance imaging (MRI). The method can detect or rule out accumulation of insoluble protein structures in the kidney caused by leucocyte cell-derived chemotaxin-2 (LECT2) protein.

In another version of the method, a detectable amount of a compound of formula (II):

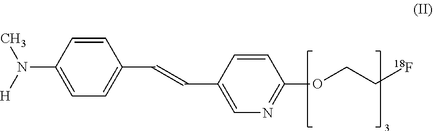

is administered to a subject. The compound is targeted to any amyloidosis in a kidney of the subject. The compound of formula (II) is also known as florbetapir ¹⁸F. An image is then acquired to detect the presence or absence of any amyloidosis in a kidney of the subject. The step of acquiring the image can be performed using an imaging method selected from positron emission tomography imaging, positron emission tomography with concurrent computed tomography imaging, and positron emission tomography with concurrent magnetic resonance imaging. The method can detect or rule out accumulation of insoluble protein structures in the kidney caused by leucocyte cell-derived chemotaxin-2 (LECT2) protein.

In another version of the method, a detectable amount of a compound of formula (III):

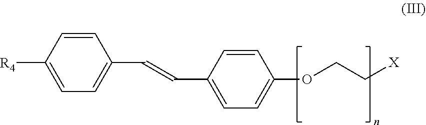

is administered to a subject. R₄ can be selected from H, OH, halogen, substituted or unsubstituted alkyl, substituted or unsubstituted alkoxy, substituted or unsubstituted amino, and substituted or unsubstituted carboxylate; n can be an integer from 0 to 10, and X can be selected from the group consisting of ³H, ¹¹C, ¹³C, ¹⁴C, ¹³N, ¹⁵O, ⁷⁵Br, ⁷⁶Br, ¹⁸F, ¹⁹F, ⁶⁸Ga, ⁸²Rb, ¹¹¹In, ¹²³I, ¹²⁵I, ¹³¹I, ²⁰¹TI, and ^{99m}Tc. The compound is targeted to any amyloidosis in a kidney of the subject. An image is then acquired to detect the presence or absence of any amyloidosis in a kidney of the subject. The step of acquiring the image can be performed using an imaging method selected from positron emission tomography imaging, positron emission tomography with concurrent computed tomography imaging, and positron emission tomography with concurrent magnetic resonance imaging. The method can detect or rule out accumulation of insoluble protein structures in the kidney caused by leucocyte cell-derived chemotaxin-2 (LECT2) protein.

In another version of the method, a detectable amount of a compound of formula (IV):

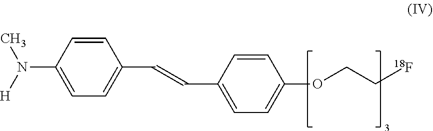

is administered to a subject. The compound is targeted to any amyloidosis in a kidney of the subject. The compound of formula (IV) is also known as florbetaben. An image is then acquired to detect the presence or absence of any amyloidosis in a kidney of the subject. The step of acquiring the image can be performed using an imaging method selected from positron emission tomography imaging, positron emission tomography with concurrent computed tomography imaging, and positron emission tomography with concurrent magnetic resonance imaging. The method can detect or rule out accumulation of insoluble protein structures in the kidney caused by leucocyte cell-derived chemotaxin-2 (LECT2) protein.

In another version of the method, a detectable amount of a compound of formula (V):

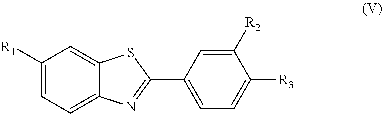

is administered to a subject. R₁, R₂ and R₃ in Formula (V) can be independently selected from H, OH, halogen, substituted or unsubstituted alkyl, substituted or unsubstituted alkoxy, substituted or unsubstituted amino, and substituted or unsubstituted carboxylate. At least one of the atoms in R₁ or R₂ or R₃ is replaced with ³H, ¹¹C, ¹³C, ¹⁴C, ¹³N, ¹⁵O, ⁷⁵Br, ⁷⁶Br, ¹⁸F, ¹⁹F, ⁶⁸Ga, ⁸²Rb, ¹¹¹In, ¹²³I, ¹²⁵I, ¹³¹I, ²⁰¹TI, and ^{99m}Tc. The compound is targeted to any amyloidosis in a kidney of the subject. An image is then acquired to detect the presence or absence of any amyloidosis in a kidney of the subject. The step of acquiring the image can be performed using an imaging method selected from positron emission tomography imaging, positron emission tomography with concurrent computed tomography imaging, and positron emission tomography with concurrent magnetic resonance imaging. The method can detect or rule out accumulation of insoluble protein structures in the kidney caused by leucocyte cell-derived chemotaxin-2 (LECT2) protein.

In another version of the method, a detectable amount of a compound of formula (VI):

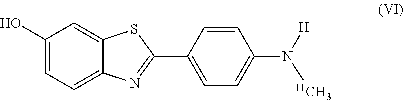 [SMILES: [11CH3]Nc1ccc(-c2nc3ccc(O)cc3s2)cc1]

is administered to a subject. The compound is targeted to any amyloidosis in a kidney of the subject. The compound of formula (VI) is also known as [N-Methyl-¹¹C]2-(4′-methylaminophenyl)-6-hydroxybenzothiazole (CAS Number 566170-04-5) or Pittsburgh compound B (PiB). An image is then acquired to detect the presence or absence of any amyloidosis in a kidney of the subject. The step of acquiring the image can be performed using an imaging method selected from positron emission tomography imaging, positron emission tomography with concurrent computed tomography imaging, and positron emission tomography with concurrent magnetic resonance imaging. The method can detect or rule out accumulation of insoluble protein structures in the kidney caused by leucocyte cell-derived chemotaxin-2 (LECT2) protein.

In another version of the method, a detectable amount of a compound of formula (VII):

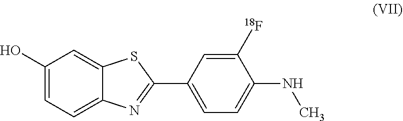 [SMILES: CNc1ccc(-c2nc3ccc(O)cc3s2)cc1[18F]]

is administered to a subject. The compound is targeted to any amyloidosis in a kidney of the subject. The compound of formula (VII) is also known as 2-[3-(¹⁸F)Fluoro-4-(methylamino)phenyl]-1,3-benzothiazol-6-ol, or flutemetamol (¹⁸F). An image is then acquired to detect the presence or absence of any amyloidosis in a kidney of the subject. The step of acquiring the image can be performed using an imaging method selected from positron emission tomography imaging, positron emission tomography with concurrent computed tomography imaging, and positron emission tomography with concurrent magnetic resonance imaging. The method can detect or rule out accumulation of insoluble protein structures in the kidney caused by leucocyte cell-derived chemotaxin-2 (LECT2) protein.

Thus, one can administer the compound of Formula (I) or Formula (II) or Formula (III) or Formula (IV) or Formula (V) or Formula (VI) or Formula (VII) as an imaging agent to a subject with presumed renal amyloidosis. One can acquire a PET image or a combined PET/CT image to detect the presence or absence of any amyloidosis in a kidney of the subject. A subject is a mammal, such as a human.

It is one advantage of the invention to provide for the use of a stilbene derivative, such as florbetapir ¹⁸F, or a phenylbenzothiazole derivative in PET imaging or PET/CT imaging to serve as a screening test for renal amyloidosis in a subject. The method can detect or rule out accumulation of insoluble protein structures in the kidney caused by leucocyte cell-derived chemotaxin-2 (LECT2) protein. The method provides a means for renal amyloidosis tissue typing in a patient before an erroneous presumed diagnosis of immunoglobulin light chain-derived amyloidosis (AL) leads to the initiation of AL chemotherapy.

These and other features, aspects, and advantages of the present invention will become better understood upon consideration of the following detailed description, drawings and appended claims.

### BRIEF DESCRIPTION OF THE DRAWINGS

FIG. 1 shows a positron emission tomography (PET) system in which an imaging agent of the present disclosure can be used.

FIG. 2 shows images of a subject obtained using positron emission tomography with computed tomography imaging (PET/CT) using florbetapir ¹⁸F.

### DETAILED DESCRIPTION OF THE INVENTION

The method of the invention exploits the ability of radiolabeled stilbene derivatives or phenylbenzothiazole derivatives to accumulate in LECT2-based amyloidosis. The method of the invention determines the presence (if any) and location of LECT2-based amyloidosis at a part (e.g., kidney) of the body of a subject. The method includes the step of administering of a detectable amount of a pharmaceutical composition including a radiolabeled stilbene derivative or a phenylbenzothiazole derivative chosen from the compounds of Formulas (I) to (VII) above to a subject. A “detectable amount” means that the amount of the detectable compound that is administered is sufficient to enable detection of accumulation of the compound in LECT2-based protein structures by a medical imaging technique. A “subject” is a mammal, preferably a human, and most preferably a human suspected of renal amyloidosis.

In vivo detection of the accumulated compound of any of Formulas (I) to (VII) in LECT2-based protein structures can be achieved by medical imaging techniques such as positron emission tomography (PET), computed tomography imaging (CT), magnetic resonance imaging (MRI), single-photon emission computed tomography (SPECT) and any combinations thereof. In the radiolabeled stilbene derivative or phenylbenzothiazole derivative chosen from the compounds of Formulas (I) to (VII) above, the type of medical imaging device is a factor in selecting a given label. For instance, the isotopes ³H, ¹¹C, ¹³C, ¹⁴C, ⁷⁵Br, ⁷⁶Br, ¹⁸F, ¹⁹F, ⁶⁸Ga, ¹¹¹In, ¹²³I, ¹²⁵I, ¹³¹I, and ^{99m}Tc are particularly suitable labels for in vivo imaging in the methods of the invention. The type of medical imaging device used will guide the selection of the isotope. For PET detection, the radiolabel will be a positron-emitting radionuclide which will annihilate to form two gamma rays which will be detected by the PET camera. For SPECT detection, the chosen radiolabel will produce minimal if any particulate emission, but will produce a large number of photons.

Concurrent use of two or more of the medical imaging techniques such as PET, CT, MRI, and SPECT can be advantageous in the method of the invention. For example, PET images can demonstrate better correlation to patient anatomy on a CT taken at the time of PET than to patient anatomy on a separate CT (usually taken before the PET image). By using a PET and CT taken back to back with the patient in the same position in the method of the invention, the risk of errors due to motion can be reduced.

For purposes of in vivo imaging, the type of detection instrument available is a major factor in selecting a given label. For instance, ¹⁹F or ¹³C are suitable for MRI; ¹⁸F, ¹¹C, ¹³N, ¹⁵O, ⁷⁵Br, ⁷⁶Br, ⁸²Rb are suitable for PET; and ¹¹¹In, ¹²³I, ¹²⁵I, ¹³¹I, ²⁹¹TI and ^{99m}Tc are suitable for SPECT imaging. ³H or ¹⁴C are suitable for in vitro imaging studies.

Administration to the subject of a pharmaceutical composition including a radiolabeled stilbene derivative or phenylbenzothiazole derivative chosen from the compounds of Formulas (I) to (VII) above for in vivo detection of the accumulated compound in LECT2-based protein structures can be accomplished intravenously, intraarterially, intrathecally, intramuscularly, intradermally, subcutaneously, or intracavitary. Dosage can vary. In one non-limiting example, dosage may vary from 0.001 μg/kg to 10 μg/kg. In another non-limiting example, the dose for florbetapir may be 370 MBq (10 mCi), maximum 50 μg mass dose, administered as a single intravenous bolus in a total volume of 10 mL or less. The injection may be followed with an intravenous flush of 0.9% sterile sodium chloride. In the method of the invention, sufficient time is allowed after administration such that the radiolabeled stilbene derivative or phenylbenzothiazole derivative can accumulate in any LECT2-based protein structures.

We have discovered that the compound of formula (II) above accumulates within LECT2-based protein structures. We have also shown that renal amyloidosis can be diagnosed using positron emission tomography using the compound of formula (II). Based on this discovery, we propose a new use of the compound of formula (I) and related radiolabeled stilbene derivatives, and a new medical indication for positron emission tomography using the compound of formula (I) and related radiolabeled stilbene derivatives, i.e., the imaging of tumors to diagnose renal amyloidosis without the need for biopsy. Positron emission tomography using the compound of formula (I) and related radiolabeled stilbene derivatives could also be used for planning treatment of ALECT2-based disease.

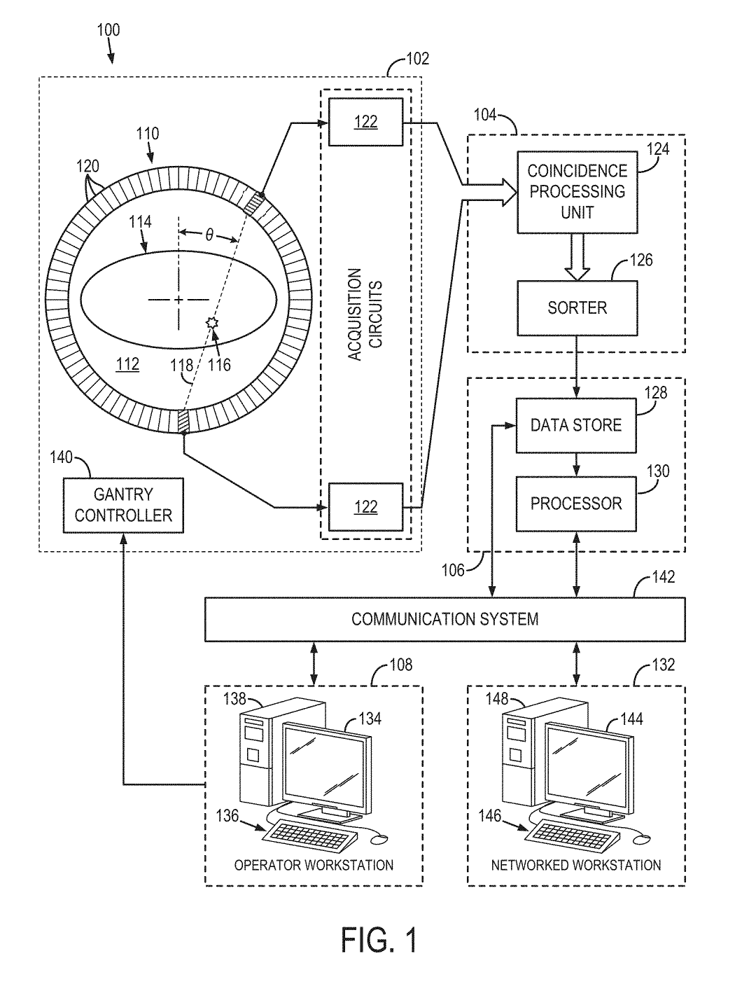

Referring now to FIG. 1, an example of a PET system 100 in which an imaging agent of the present disclosure can be used is illustrated. The PET system 100 includes an imaging hardware system 102, a data acquisition system 104, a data processing system 106, and an operator workstation 108.

The imaging hardware system 102 generally includes a PET scanner having a radiation detector ring assembly 110 that is centered about the bore 112 of the PET scanner. The bore 112 of the PET scanner is sized to receive a subject 114 for examination. Prior to imaging, the subject 114 is administered a radioisotope, such as one of the imaging agents of the present disclosure which includes a positron emitter.

Positrons are emitted by the positron emitter as it undergoes radioactive decay. These positrons travel a short distance before encountering electrons at which time the positron and electron annihilate. The positron-electron annihilation event 116 generates two photons that travel in opposite directions along a generally straight line 118.

The radiation detector ring assembly 110 is formed of multiple radiation detectors 120. By way of example, each radiation detector 120 may include one or more scintillators and one or more photodetectors. Examples of photodetectors that may be used in the radiation detectors 120 include photomultiplier tubes or avalanche photodiodes. The radiation detectors 120 are thus configured to produce a signal responsive to the photons generated by annihilation events 116. The signal responsive to the detection of a photon is communicated to a set of acquisition circuits 122. The acquisition circuits 122 receive the photon detection signals and produce signals that indicate the coordinates of each detected photon, the total energy associated with each detected photon, and the time at which each photon was detected. These data signals are sent the data acquisition system 104 where they are processed to identify detected photons that correspond to an annihilation event 116.

The data acquisition system 104 generally includes a coincidence processing unit 124 and a sorter 126. The coincidence processing unit 124 periodically samples the data signals produced by the acquisition circuits 122. The coincidence processing unit 124 assembles the information about each detected annihilation event 116 into a set of numbers that indicate when the event took place and the position in which the event was detected. This event data is then processed by the coincidence processing unit 124 to determine if any two detected photons correspond to a valid coincidence event.

As one example, the coincidence processing unit 124 may determine if any two detected photons are in coincidence as follows. First, the times at which two photons were detected should be within a predetermined time window, for example, within 0-12 nanoseconds of each other, such as within a time window of 3-4 nanoseconds. Second, the locations at which the two photons were detected should lie on a line 118 that passes through the field of view in the PET scanner bore 112. Each valid coincidence event represents the line 118 connecting the two radiation detectors 120 along which the annihilation event 116 occurred, which is referred to as a line-of-response. The data corresponding to each identified valid coincidence event is stored as coincidence data, which represents the near-simultaneous detection of photons generated by an annihilation event 116 and detected by a pair of radiation detectors 120.

The coincidence data are communicated to a sorter 126 where the coincidence events are grouped into projection images, which may be referred to as sinograms. The sorter 126 sorts each sinogram by the angle of each view, which may be measured as the angle, of the line-of-response 118 from a reference direction that lies in the plane of the detector ring assembly 102. For three-dimensional images, the sorter 126 may also sort the sinograms by the tilt of each view. The sorter 126 may also process and sort additional data corresponding to detected photons, including the time at which the photons were detected and their respective energies.

After sorting, the sinograms are provided to the data processing system 106 for processing and image reconstruction. The data processing system 106 may include a data store 128 for storing the raw sinogram data. Before image reconstruction, the sinograms may undergo preprocessing to correct for random and scatter coincidence events, attenuation effects, and other sources of error. The stored sinogram data may thus be processed by a processor 130 located on the data processing system 106, by the operator workstation 108, or by a networked workstation 132.

The operator workstation 108 typically includes a display 134; one or more input devices 136, such as a keyboard and mouse; and a processor 138. The processor 138 may include a commercially available programmable machine running a commercially available operating system. The operator workstation 108 provides the operator interface that enables scan prescriptions to be entered into the PET system 100. In general, the operator workstation 108 may be in communication with a gantry controller 140 to control the positioning of the detector ring assembly 110 with respect to the subject 114 and may also be in communication with the data acquisition system 104 to control operation of the imaging hardware system 102 and data acquisition system 104 itself.

The operator workstation 108 may be connected to the data acquisition system 104 and data processing system 106 via a communication system 142, which may include any suitable network connection, whether wired, wireless, or a combination of both. As an example, the communication system 142 may include both proprietary or dedicated networks, as well as open networks, such as the internet.

The PET system 100 may also include one or more networked workstations 132. By way of example, a networked workstation 132 may include a display 144; one or more input devices 146, such as a keyboard and mouse; and a processor 148. The networked workstation 132 may be located within the same facility as the operator workstation 108, or in a different facility, such as a different healthcare institution or clinic.

The networked workstation 132, whether within the same facility or in a different facility as the operator workstation 108, may gain remote access to the data processing system 106 or data store 128 via the communication system 142. Accordingly, multiple networked workstations 132 may have access to the data processing system 106 and the data store 128. In this manner, sinogram data, reconstructed images, or other data may be exchanged between the data processing system 106 or the data store 128 and the networked workstations 132, such that the data or images may be remotely processed by a networked workstation 132. This data may be exchanged in any suitable format, such as in accordance with the transmission control protocol, the internet protocol, or other known or suitable protocols.

Concurrent use of PET and CT can be advantageous in the method of the invention. For example, PET images can demonstrate better correlation to subject anatomy on a CT taken at the time of PET than to subject anatomy on a separate CT (usually taken before the PET image). By using PET and CT taken back to back with the subject in the same position in the method of the invention, the risk of errors due to motion can be reduced.

Thus, in one embodiment, the invention provides a method for imaging a subject to detect or rule out renal amyloidosis in the subject. The method comprises: (a) administering a compound of formula (I) to a region of interest within the subject,

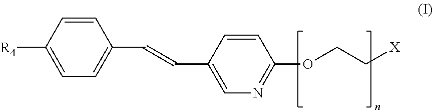

wherein R₄ is selected from the group consisting of H, OH, halogen, substituted or unsubstituted alkyl, substituted or unsubstituted alkoxy, substituted or unsubstituted amino, and substituted or unsubstituted carboxylate,

wherein n is an integer from 0 to 10,

wherein X is selected from the group consisting of ³H, ¹¹C, ¹³C, ¹⁴C, ¹³N, ¹⁵O, ⁷⁵Br, ⁷⁶Br, ¹⁸F, ¹⁹F, ⁶⁸Ga, ⁸²Rb, ¹¹¹In, ¹²³I, ¹²⁵I, ¹³¹I, ²⁰¹TI, and ^{99m}Tc; and

(b) acquiring data from the region of interest of the subject using a positron emission tomography system; and (c) reconstructing an image of the region of interest from the acquired data, wherein the region of interest includes a kidney of the subject. Step (b) may comprise acquiring the data using a positron emission tomography system with computed tomography imaging. The method can detect or rule out accumulation of insoluble protein structures in the kidney caused by leucocyte cell-derived chemotaxin-2 (LECT2) protein. In the region of interest, normal background activity may be observed in the heart of the subject thereby distinguishing LECT2 amyloidosis from transthyretin-related amyloidosis (ATTR) and immunoglobulin light chain-derived amyloidosis (AL).

In another embodiment, the invention provides a method for imaging a subject to detect or rule out renal amyloidosis in the subject. The method comprises: (a) administering a compound of formula (II) to a region of interest within the subject,

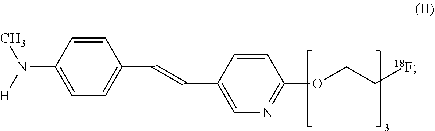

(b) acquiring data from the region of interest of the subject using a positron emission tomography system; and (c) reconstructing an image of the region of interest from the acquired data, wherein the region of interest includes a kidney of the subject. Step (b) may comprise acquiring the data using a positron emission tomography system with computed tomography imaging. The method can detect or rule out accumulation of insoluble protein structures in the kidney caused by leucocyte cell-derived chemotaxin-2 (LECT2) protein. In the region of interest, normal background activity may be observed in the heart of the subject thereby distinguishing LECT2 amyloidosis from transthyretin-related amyloidosis (ATTR) and immunoglobulin light chain-derived amyloidosis (AL). Alternatively, Formula (III) or Formula (IV) or Formula (V) or Formula (VI) or Formula (VII) may be used in this embodiment of the method of the invention.

### EXAMPLES

The following Examples have been presented in order to further illustrate the invention and are not intended to limit the invention in any way.

### Example 1

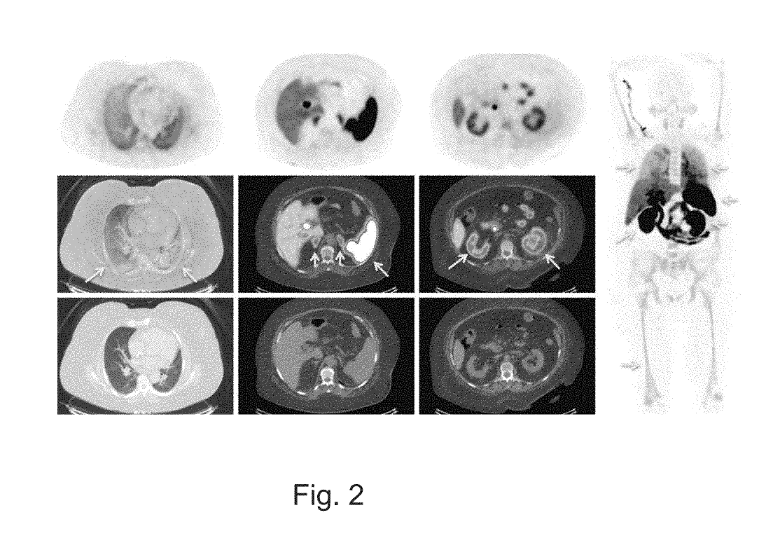

A 69-year-old Mexican female with end stage renal disease on haemodialysis secondary to biopsy-confirmed leucocyte cell-derived chemotaxin-2 (LECT2) amyloidosis underwent a positron emission tomography/computed tomography (PET/CT) scan with florbetapir ¹⁸F. In FIG. 2, green arrows highlight diffuse intense activity above normal background in the lungs (maximum standardized uptake value [SUVₘₐₓ] 10·0), spleen (SUVₘₐₓ 40·6), adrenal glands (SUVₘₐₓ 12·3), kidneys (SUVₘₐₓ 16·3) and bone marrow (images). These organs normally have low activity on florbetapir PET/CT. The activity pattern is consistent with pathological data on the distribution of amyloidosis in this disease. Unlike transthyretin-related amyloidosis (ATTR) and immunoglobulin light chain-derived amyloidosis (AL), there is normal background activity in the heart of this patient, which is consistent with autopsy data showing cardiac sparing in LECT2 amyloidosis. A remarkable finding in this patient is the uptake in the kidney because she makes little urine and is on chronic haemodialysis.

### Prophetic Example A

One would administer the compound of Formula (IV) to a patient with presumed LECT2 amyloidosis. The compound of formula (IV) is also known as 4-{(E)-2-[4-(2-{2-[2-(18F)Fluoroethoxy]ethoxy}ethoxy)phenyl]vinyl}-N-methylaniline, or florbetaben, or AV-1, or BAY94-9172. One would acquire a combined PET/CT image to detect the presence or absence of any LECT2-based amyloidosis in the patient. One would envision that PET/CT data would confirm LECT2-based amyloidosis uptake of Formula (IV). One would envision that other stilbene derivatives would confirm LECT2-based amyloidosis uptake.

### Prophetic Example B

One would administer the compound of Formula (VI) to a patient with presumed LECT2 amyloidosis. The compound of formula (VI) is also known as [N-Methyl-¹¹C]₂-(4′-methylaminophenyl)-6-hydroxybenzothiazole (CAS Number 566170-04-5) or Pittsburgh compound B (PiB). It can be synthesized using the methods described in U.S. Pat. No. 7,270,800. One would acquire a combined PET/CT image to detect the presence or absence of any LECT2-based amyloidosis in the patient. One would envision that PET/CT data would confirm LECT2-based amyloidosis uptake of Formula (VI). One would envision that other phenylbenzothiazole derivatives would confirm LECT2-based amyloidosis uptake.

### Prophetic Example C

One would administer the compound of Formula (VII) to a patient with presumed LECT2 amyloidosis. The compound of formula (VII) is also known as 2-[3-(¹⁸F)Fluoro-4-(methylamino)phenyl]-1,3-benzothiazol-6-ol, or flutemetamol. One would acquire a combined PET/CT image to detect the presence or absence of any LECT2-based amyloidosis in the patient. One would envision that PET/CT data would confirm LECT2-based amyloidosis uptake of Formula (VII). One would envision that other phenylbenzothiazole derivatives would confirm LECT2-based amyloidosis uptake.

Thus, the invention provides a method for detecting or ruling out renal amyloidosis in a subject. The use of a stilbene derivative, such as florbetapir ¹⁸F or florbetaben, or a phenylbenzothiazole derivative, such as PiB or flutemetamol, in PET/CT imaging can serve as a screening test for renal amyloidosis in a subject.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A method for detecting or ruling out renal amyloidosis in a subject, the method comprising:
(a) administering to a subject a detectable amount of a compound of formula (I): 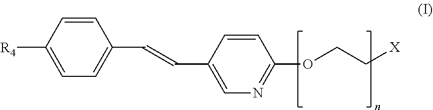
wherein the compound is targeted to a kidney of the subject,
wherein R₄ is selected from the group consisting of H, OH, halogen, substituted or unsubstituted alkyl, substituted or unsubstituted alkoxy, substituted or unsubstituted amino, and substituted or unsubstituted carboxylate,
wherein n is an integer from 0 to 10,
wherein X is selected from the group consisting of ³H, ¹¹C, ¹³C, ¹⁴C, ¹³N, ¹⁵O, ⁷⁵Br, ⁷⁶Br, ¹⁸F, ¹⁹F, ⁶⁸Ga, ⁸²Rb, ¹¹¹In, ¹²³I, ¹²⁵I, ¹³¹I, ²⁰¹TI, and ^{99m}Tc; and
(b) acquiring an image to detect the presence or absence of any amyloidosis in a kidney of the subject.

2. The method of claim 1 wherein:
R₄ is methylamino or dimethylamino.

3. The method of claim 1 wherein:
R₄ is methylamino, and
X is ¹⁸F.

4. The method of claim 1 wherein:
step (b) comprises acquiring the image using an imaging method selected from positron emission tomography imaging, positron emission tomography with computed tomography imaging, and positron emission tomography with magnetic resonance imaging.

5. The method of claim 1 wherein:
step (b) comprises acquiring the image using positron emission tomography imaging.

6. The method of claim 1 wherein:
step (b) comprises acquiring the image using positron emission tomography with computed tomography imaging.

7. The method of claim 1 wherein:
the subject makes little urine.

8. The method of claim 1 wherein:
the subject is on chronic haemodialysis.

9. The method of claim 1 wherein:
the method detects or rules out accumulation of insoluble protein structures in the kidney caused by leucocyte cell-derived chemotaxin-2 (LECT2) protein.

10. A method for detecting or ruling out renal amyloidosis in a subject, the method comprising:
(a) administering to a subject a detectable amount of a compound of formula (II): 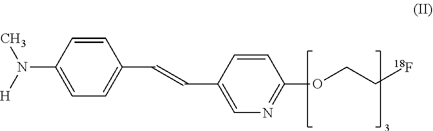
wherein the compound is targeted to a kidney of the subject; and
(b) acquiring an image to detect the presence or absence of any amyloidosis in a kidney of the subject.

11. The method of claim 10 wherein:
step (b) comprises acquiring the image using an imaging method selected from positron emission tomography imaging, positron emission tomography with computed tomography imaging, and positron emission tomography with magnetic resonance imaging.

12. The method of claim 10 wherein:
step (b) comprises acquiring the image using positron emission tomography imaging.

13. The method of claim 10 wherein:
step (b) comprises acquiring the image using positron emission tomography with computed tomography imaging.

14. The method of claim 10 wherein:
the method detects or rules out accumulation of insoluble protein structures in the kidney caused by leucocyte cell-derived chemotaxin-2 (LECT2) protein.

15. A method for detecting or ruling out renal amyloidosis in a subject, the method comprising:
(a) administering to a subject a detectable amount of a compound of formula (III): 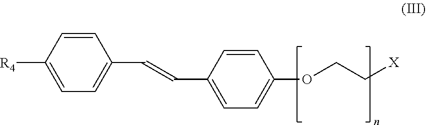
wherein R₄ is selected from H, OH, halogen, substituted or unsubstituted alkyl, substituted or unsubstituted alkoxy, substituted or unsubstituted amino, and substituted or unsubstituted carboxylate,
wherein n is an integer from 0 to 10,
wherein X is selected from the group consisting of ³H, ¹¹C, ¹³C, ¹⁴C, ¹³N, ¹⁵O, ⁷⁵Br, ⁷⁶Br, ¹⁸F, ¹⁹F, ⁶⁸Ga, ⁸²Rb, ¹¹¹In, ¹²³I, ¹²⁵I, ¹³¹I, ²⁰¹TI, and ^{99m}Tc, and
wherein the compound is targeted to a kidney of the subject; and
(b) acquiring an image to detect the presence or absence of any amyloidosis in a kidney of the subject.

16. The method of claim 15 wherein:
R₄ is methylamino,
n is 3, and
X is ¹⁸F.

17. A method for detecting or ruling out renal amyloidosis in a subject, the method comprising:
(a) administering to a subject a detectable amount of a compound of formula (V): 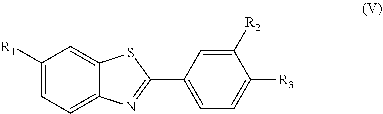
wherein R₁, R₂ and R₃ are independently selected from H, OH, halogen, substituted or unsubstituted alkyl, substituted or unsubstituted alkoxy, substituted or unsubstituted amino, and substituted or unsubstituted carboxylate,
wherein at least one of the atoms in R₁ or R₂ or R₃ is replaced with ³H, ¹¹C, ¹³C, ¹⁴C, ¹³N, ¹⁵O, ⁷⁵Br, ⁷⁶Br, ¹⁸F, ¹⁹F, ⁶⁸G, ⁸²Rb, ¹¹¹In, ¹²³I, ¹²⁵I, ¹³¹I, ²⁰¹TI, and ^{99m}Tc, and
wherein the compound is targeted to a kidney of the subject; and
(b) acquiring an image to detect the presence or absence of any amyloidosis in a kidney of the subject.

18. The method of claim 17 wherein:
R₁ is OH,
R₂ is H,
R₃ is methylamino, and
one of the atoms in R₃ is replaced with ¹¹C.

19. The method of claim 17 wherein:
R₁ is OH,
R₂ is ¹⁸F, and
R₃ is methylamino.
